(19) United States
(12) Patent Application Publication
(10) Pub. No.: US 2010/0042139 A1
(43) Pub. Date: Feb. 18, 2010

(54) Device for reshaping bones

(76) Inventor: Norina Honegger, Herrliberg (CH)

Correspondence Address: FISH & ASSOCIATES, PC;ROBERT D. FISH, 2603 Main Street, Suite 1000, Irvine, CA, 92614-6232, US

(21) Appl. No.: 12/311,582

(22) PCT Filed: Oct. 1, 2007
(86) PCT No.: PCT/EP2007/060403

§ 371 Date: Oct. 23, 2009

(30) Foreign Application Priority Data
Oct. 2, 2006 (EP) 06121632.1

(51) Int. Cl. A61F 5/08

(52) U.S. Cl. 606/204.45

(57) ABSTRACT

The invention relates to a device (1) for reshaping bones, particularly nasal bones, comprising: a molding device (2) with a pressing surface (2d) for placing onto the skin, and a holding device (3), which can be connected to the molding device (2), wherein the holding device (3) and the molding device (2) are designed to interact with each other such that the molding device (2) can be placed onto the skin at least on the pressing surface (2d) while applying a contact pressure.

The invention relates to a device for reshaping bones and for the influencing of bone growth, in particular of the nasal bone. The invention furthermore relates to a method for manufacturing a device for reshaping bones.

### PRIOR ART

The shape of the nose is of central importance for an attractive facial profile. The nose is ideally embedded extending harmoniously in the facial features. If, however, the nose has an idiosyncratic shape and/or a disproportionate size, the nose inevitably becomes an attention-getter and thereby becomes the dominant part of the face.

The shape of the nose can today usually be corrected in the long term by a comparatively small, but nevertheless demanding, surgical procedure. The nose is made smaller in more than 80 percent of the procedures and larger in the remaining procedures.

The nose is substantially made up of osseous and cartilaginous portions which form a pyramid-like framework beneath the covering skin of the nose. The nasal septum is located perpendicularly at the center and is cartilaginous in the front portion and osseous in the rear portion. The purpose of nose operations is to design this framework such that the outer shape of the nose is changed by the covering skin. Rhinoplasty can make the nose smaller or larger, can correct the shape of the bridge of the nose as well as of the nose inlet with the alae of the nose and the nostrils, and can change the length of the nose as well as the angle between the nose and the upper lip. The procedure is in particular carried out for cosmetic reasons for the harmonization of the nose and of the total face (cosmetic rhinoplasty). So that the procedure does not leave any visible traces, the skin incisions are applied to the interior of the nose or at the septum. The physician remodels the bone framework and cartilage framework of the nose in an advantageous manner in dependence on the objective through this access. The nose can thus, for example, be made smaller, narrower or made straight. Such a correction of the nose should only be carried out after the end of bone growth, that is approximately from the age of 16 or 17.

This known method has the disadvantages that a demanding surgical procedure is required with all its associated risks, that the procedure is very expensive and is not usually financed by health insurance plans, and that the method is only accessible to a limited population for financial reasons.

Reference DE 321737 discloses a nose shaper which is made up of an enveloping body with an adjustable pressure part. The nose shaper is fixed to the head and the pressure part is intended to exert a pressure onto the nose. This nose shaper has the disadvantages that the exact position of the pressure part cannot be determined precisely, that the position of the pressure part may change during the wearing of the nose shaper, and that the nose shaper is extremely unpleasant to wear and is therefore not suitable for longer and repeated wear, in particular also not during sleep.

Reference DE 811 255 discloses an eyewear-like device for nose shaping. This device has the disadvantage that the position of the pressure part can change during wear, that the maximum compressive force which can be generated by the pressure part is too small, and that the nose shaper is extremely unpleasant to wear and is therefore not suitable for longer and repeated wear, in particular also not during sleep.

All known nose shapers have the disadvantage that their position can change during wear, that is they slip with respect to the nose. In addition, wearing is unpleasant and annoying.

### REPRESENTATION OF THE INVENTION

It is the object of the invention to propose a device, as well as a method for manufacturing the device, which allows a cost-effective and safe reshaping of bones and in particular a correction of the shape of the nose.

This object is satisfied by a device for reshaping bones or cartilage having the features of claim 1. Dependent claims 2 to 14 relate to further advantageous embodiments. The object is further satisfied using a method for manufacturing the device in accordance with the invention having the features of claim 15. The object is further satisfied using a kit including the features of claims 16 to 20.

The object is in particular satisfied by a device for reshaping bones and for influencing the growth of bones or cartilage, in particular nasal bones, including a shaping device having a pressing surface designed for placing onto the skin and including a holding device which can be connected to the shaping device, with the holding device as well as the shaping device being made to cooperate such that the shaping device can contact the skin at least at the pressing surface while exerting a pressing force.

The object is further in particular satisfied by a device for reshaping bones, in particular nasal bones, including a shaping device having a pressure part having a pressing surface designed for placing onto the skin and a holding device which can be connected to the shape device, with the holding device as well as the shaping device being made to cooperate such that the shaping device can contact the skin at least at the pressing surface while exerting a pressing force, with the shaping device including a carrier having a support surface, and with the carrier being made to extend such that the contact surface follows the outer contour of the root of the nose at least along a part portion and can lie over the contact surface on the face.

In the present specification, bones are also understood as bone-like structures or cartilage.

It has been found that human bones are subject to a continuous modification process during their whole lives. On the normal modification of the bone, there is a balance between osteoclastic and osteoblastic activity. A long-term load on the bone, however, results in adaptation processes such as thickening of the corticalis and alignment of the spongiosa trabeculae. If the individual load tolerance is exceeded by repeated submaximal similar stimuli, structural changes and modification processes take place in the region of mechanical load peaks. The present invention exploits this effect in that such a pressure is generated on specific bone regions that a strain arises in this bone which influences the shape of the bone or cartilage. For this purpose, a force is exerted onto the skin located above the named bone region, said force propagating over the skin onto the bone so that the shape of the bone is influenced by the shaping device in accordance with the invention. The device in accordance with the invention is preferably used on children in the growth phase since the arrangement of the bone, in particular of the nasal bone, is relatively easy to influence in this phase. The device in accordance with the invention is, however, also usable for adults. The device in accordance with the invention is preferably used for several hours a day, for example during sleep.

In order, for example, to correct or reshape the shape of the nose the device in accordance with the invention is put on before going to sleep in that the shaping device is placed onto the nose and the shaping device is fixed to the head using a holding device. The shaping device has pressing surfaces which are oriented, arranged and designed such that a pressing force is applied onto a predetermined point of the nasal bone during sleep.

The shape of the nasal bone changes or the growing nasal bone approximates a preset shape by usually several months up to several years of wear of the device in accordance with the invention during sleep. This is preferably used during the growth phase so that the nasal bone has the desired nasal bone shape and also retains it after the end of bone growth, that is after the age of 16 or 17.

In an advantageous embodiment, the device has a carrier which forms a support surface. The support surface preferably extends at least over a part section along the root of the nose and lies on the face in this respect, preferably with only a small or negligibly small pressing force. The support surface designed in this manner has the consequence that the device has a relatively precisely defined position and hardly slips with respect to the nose. This is of particular importance especially on long-term wear and wear during sleep. In a particularly advantageous aspect, the carrier can be fixed to the head via a first holding device, with the pressing part being able to be fixed to the head via an additional second holding device.

This aspect has the advantage that the position of the device with respect to the nose is substantially determined by the carrier and the holding device, whereas the precise position of the force introduction is determined by the position of the pressing part and by the magnitude of the introduced force through the second holding device. This aspect has the advantage that the comfort in wear of the device is felt to be pleasant, that hardly any slipping occurs between the device and the nose so that the location of the force introduction into the nose is reproducible and constant in the long term, and in that the magnitude of the force exerted onto the nose is adjustable via the second holding device. The adjustability of the exerted force or of the force effected onto the nose is of decisive importance for long-term comfort of wear since the subjective feelings of individual persons is very different and can even change within an individual. The adjustability of the force acting on the nose therefore has the advantage that the device can always be fixed to or adjusted at the head such that any unpleasant wearing comfort can be avoided or reduced. It can thereby in particular be prevented that the nose starts to hurt in an unpleasant manner. A long-term wearing of the device can additionally thereby be ensured.

In a particularly advantageous aspect, the device is made in one piece, in particular as a light plastic part.

In a further particularly advantageous embodiment, the device is made as a kit including a plurality of patch-like covers which can be stuck into the shaping device in order thereby to form the pressing surface. The kit includes a plurality of covers which in particular differ with respect to thickness and/or area so that a corresponding cover can be selected and stuck into the shaping device in dependence on the desired pressing surface.

The device in accordance with the invention will be explained in detail in the following for the example of the reshaping of the nose. The device in accordance with the invention is, however, suitable for the reshaping of almost any human bone, for example also for the reshaping of the chin or of the cheekbones. The invention will be described in the following with reference to embodiments.

### BRIEF DESCRIPTION OF THE DRAWINGS

The drawings used to explain the embodiments show:

FIG. 1 an installed device for reshaping bones;

FIG. 2 the device shown in FIG. 1 in detail;

FIG. 3 a longitudinal section through the one-piece shaping device shown in section in FIG. 2;

FIG. 4 a further embodiment of a shaping device;

FIG. 5 a longitudinal section through the shaping device shown in FIG. 4 and placed onto a nose;

FIG. 15 a further embodiment of a pressing part;

FIG. 16 a further embodiment of a shaping device;

FIG. 17 a cross-section through a further embodiment of a pressing part;

FIG. 18 a device for manufacturing the device for reshaping bones;

FIG. 19 a further embodiment of a one-piece shaping device;

FIG. 20 a section through the shaping device shown in FIG. 19 along the line A-A;

FIG. 21 a side view of a further embodiment of a shaping device;

FIG. 22 a longitudinal section through the shaping device shown in FIG. 21;

FIG. 23 a plan view of a pressing part from the inside;

FIGS. 24a-24d differently designed covers;

FIGS. 25a-25c cross-sections through differently designed covers;

FIG. 26 a cross-section through a shaping device;

FIG. 27 a further embodiment of a one-piece shaping device;

FIG. 28 a side view of a further embodiment of a shaping device;

FIG. 29 a cross-section through a shaping device.

Generally, the same parts are provided with the same reference numerals in the drawings.

### WAYS OF PERFORMING THE INVENTION

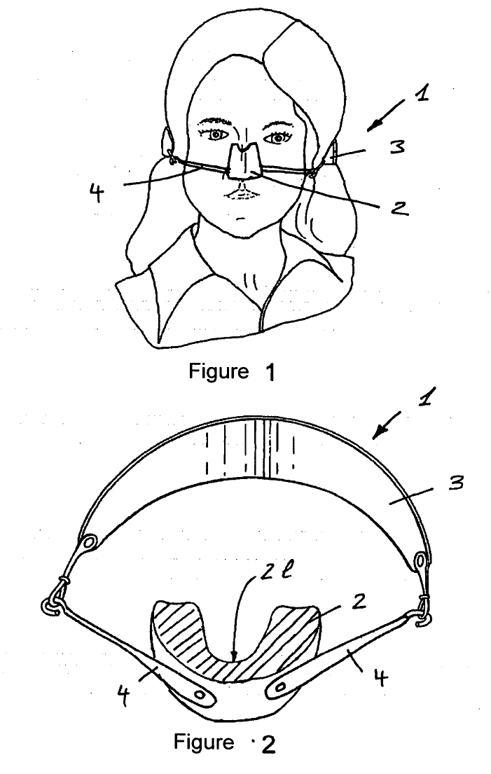

FIG. 1 shows a device 1 for reshaping nasal boned and for influencing the shape of the nose fixed to the head of a teenager. The device includes a shaping device 2 which is connected via a connection means 4 to a holding device 3 extending around the head at the rear. This device 1 is usually fixed to the head overnight so that the shaping device 2 exerts a pressing force onto the skin and the nasal bone disposed thereunder for approximately eight hours, for example.

FIG. 2 shows the device 1 shown in FIG. 1 in detail. The shaping device 2 is shown in a cross-section so that the recess 21 designed for the reception of the nose can be seen. The shaping device 2 is made in one piece and is thus made up from a single part. The holding device 3 is preferably elastic and the connection means 4 are preferably made rigid so that the shaping device 2 is pressed against the nose with a sufficiently large force and remains slip-proof, in particular during sleep. The holding device 3 can be configured in different manners to be secured to the head. For example, the holding device 3 can also have two or three bands running behind the head to effect a better grip.

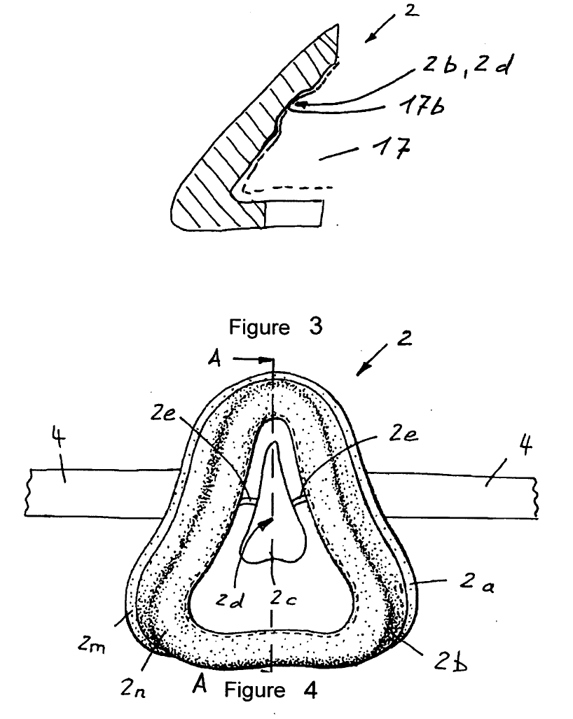

FIG. 3 shows a longitudinal section through the shaping device 2 shown in FIG. 2. The extent of the nose 17 is shown only schematically and in dashed lines. The nose has a bump 17b. The shaping device 2 has at least one support surface 2b with a pressing surface 2d for placing onto the skin so that the pressing force exerted by the shaping device 2 preferably takes place to a large part via the pressing surface 2d. The support surface 2b is therefore designed with respect to alignment and/or size and/or surface extent such that the force generated via the pressing surface 2d acts on a specific skin point and thus on a predetermined bone region 17b to influence its shape and/or growth. The pressing surface 2d can be arranged and configured in very many different ways and can be made up, for example, of two or more part surfaces, or can extend, for example, along the total length of the bridge of the nose or along the side contours. The arrangement, number, size and alignment of the pressing surfaces 2d are determined by the position of the bone region or of the cartilage region whose shape and/or growth should be influenced. It is therefore also important that the shaping device 2 can be placed onto the nose arranged reproducibly the same or similarly over preferably a larger number of applications so that the pressing force is exerted onto the same bone regions. It can therefore prove to be advantageous to provide the shaping device 2 with a recess 21 designed such that the shaping device 2 can be placed onto the nose in a more-or-less self-centering manner. The shaping device 2 is produced, for example, of plastic, with the shaping device 2 also being able to have locally different elasticities.

In an advantageous embodiment, the shaping device 2 includes, as shown in FIG. 4, a carrier 2a with an anatomically designed support surface 2b, with the carrier 2a being connected to the connection means 4. The carrier 2a is made, for example, of a metallic frame 2m and of an elastic plastic 2n which forms the support surface 2b. In the embodiment shown, the extent of the inner contour of the carrier 2a corresponds to the extent of the outer contour of the root of the nose so that the carrier 2a can be applied to the face such that the carrier 2a surrounds the nose and contacts the face via the support surface 2b in the region of the root of the nose. This large area application of the carrier 2a, which is determined by the position of the nose, has the advantage that the carrier 2a can be applied reproducibly the same and in a slip-proof manner or substantially in a slip-proof manner, in a similar manner as shown in FIG. 1. The carrier 2, or its metal frame 2m, thus forms a base part to which one or more pressing parts 2c can be fastened. In the embodiment shown, a pressing part 2c is fixedly connected to the carrier 2a via two holding means 2e made as webs. The pressing part 2c has a pressing surface 2d. A plurality of pressing parts 2c can also be connected to the carrier 2a. The pressing part 2c could also only be connected to the carrier 2a with a single holding means 2e. The pressing parts 2c could also be arranged displaceably and/or releasably and fixably again with respect to the holding means 2e. The carrier 2a shown in FIG. 4 can thus be equipped with pressing parts 2c in the most varied manners.

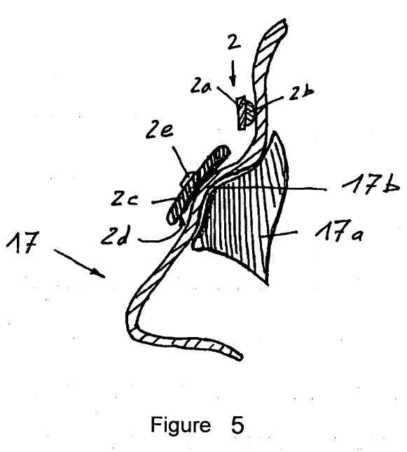

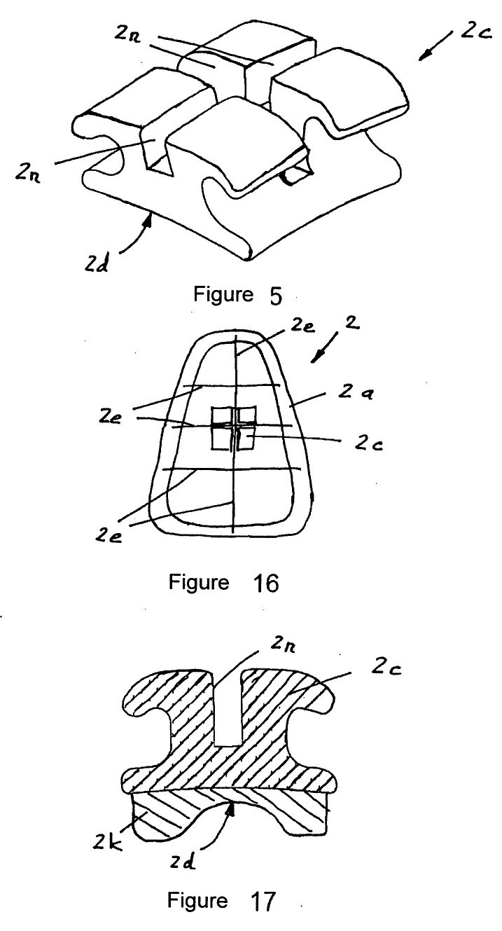

FIG. 5 schematically shows a longitudinal section through a nose 17 with a nasal bone 17a. The shaping device 2 shown in FIG. 4 is placed onto the nose 17. The pressing part 2c with the pressing surface 2d contacts the bump of the nose 17b and exerts a pressing force onto the bump of the nose 17b or onto the nasal bone 17a. The pressing part 2c or the pressing surface 2d can, as already described using FIG. 3, be designed and arranged in a plurality of possibilities depending on the respective anatomical shape of the nose. A plurality of pressing surfaces 2d or also a plurality of pressing parts 2c can also be provided.

FIG. 2 shows an embodiment of a carrier 2a with a round metallic frame or with a plastic frame 2m as well as with an elastic plastic 2n surrounding the frame 2m. The metallic frame or the plastic frame 2m can be made flexible in an advantageous embodiment so that the extent of the frame 2m can be adapted, for example, corresponding to the anatomical extent of the nose or of the root of the nose. Each limb of the carrier 2a can also be connected to a holding device 3 via a respective so-called facial arch 5. The two facial arches 5 can be fixedly connected to one another at their points of intersection.

In an advantageous embodiment, a kit is available for the adaptation of the shaping device 2a, said kit including a plurality of carriers 2a of different sizes and/or shapes as well as a plurality of pressing parts 2c, preferably also of different sizes and/or designs, as well as a plurality of different facial arches 5. Such a kit allows a plurality of differently designed shaping devices 2 to be formed adapted individually to the anatomy of a person.

As shown in a plan view in FIG. 16, a plurality of holding means 2e, which are releasably or fixedly connected to the carrier 2a, could also be arranged at the carrier 2a. The holding means 2e can also be made in grid form with, for example, fixedly connected intersection points. The holding means 2e are advantageously made as a thin wire of, for example, 1 mm or 0.5 mm to 1.5 mm diameter, with the grid being arched such that it extends spaced apart with respect to the nose when the shaping device 2 is put on. Such a carrier 2a with a holding means 2e can have a very low weight. Advantageously, as shown in FIG. 15, a pressing part 2c is used which has crossing grooves 2n. At intersecting holding means 2e, this pressing part 2c is advantageously, as shown in FIG. 16, connected to said holding means, e.g. by soldering or adhesive bonding, so that the pressing part 2c is held in an exactly defined position in the grid or in the shaping device 2. If necessary, the pressing part 2c can also be replaced by another pressing part 2c. A pressing part 2c would, however, also be suitable without crossing grooves, said pressing part being able to be secured to the grid. A plurality of pressing parts 2c could also be fastened to the grid.

FIG. 17 shows a cross-section through a further embodiment of a pressing part 2c. In contrast to the embodiment shown in FIG. 15, the pressing part 2c in accordance with FIG. 17 additionally has a shaping part 2k which forms the pressing surface 2d.

FIG. 18 schematically shows a device 10 for manufacturing the shaping device 2. In the embodiment shown, the preparation of a shaping device 2 for reshaping the nose is shown. The same device 10 is naturally also suitable for the preparation of shaping devices 2 for other body parts. The shape of the nose is scanned using a 3-dimensional scanner 11 and the data are transmitted to a server 12. A 3-dimensional data model of the measured shape of the nose is stored in the memory 14. A 3-dimensional data model of the desired nasal shape can thereupon be stored with the help of an interactive process 17. The server has access to a database 15 in which the data of a plurality of shaping devices 2, carriers 2a, pressing parts 2c, etc. are stored. The processing module 13 stored on the server 12 is able to propose the design of a suitable shaping device 2 based on the named available data. The total shaping device 2 can be composed, for example, on the basis of standard components. In a preferred embodiment, the surface extent of the shaping part 2k of each pressing part 2c is individually designed in that, for example, the server 12 is connected to a machine 16 which permits a shaping part 2k with an individually extending surface 21 to be manufactured. In an advantageous embodiment, the pressing parts 2c in their base form are provided with a shaping part 2k of constant thickness. This shaping part 2k can be individually machined in the machine 16 so that the shaping part 2k made in this way has an individually extending surface 21 with at least one pressing surface 2d. The at least one pressing part 2c is thereupon fastened to the carrier 2a so that the shaping device 2 is formed which can thereupon be placed onto the nose with a prestressing force to influence its shape.

The method for manufacturing a shaping device includes the following steps in a preferred embodiment:

a) contact-free scanning of the skin surface to be modified and preparing a digital model of the extent of the skin surface of the body position to be modified;

b) obtaining a digital model of the extent of the bone disposed thereunder;

c) fixing the desired extent of the skin surface of the body position after the modification;

d) fixing the regions of the bones to be modified;

e) determining at least one pressing surface 2d with respect to at least one parameter from the group of size, alignment, surface extent;

f) and manufacturing the shaping device 2 including the pressing surface 2d.

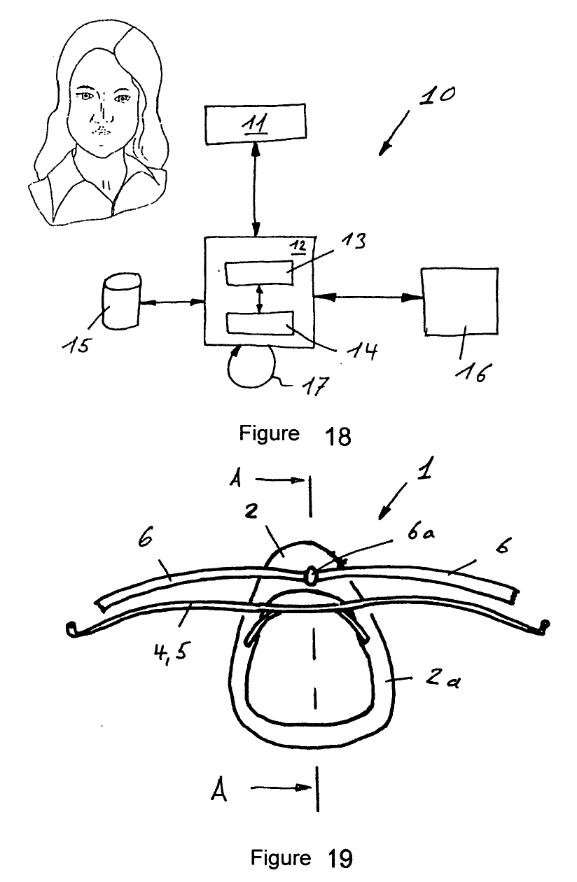

FIG. 19 shows a very simple shaping device 2 made from plastic with a carrier 2a configured to run along the root of the nose. The device 1 includes two holding devices 4, 6, a facial arch 5 which forms the first holding means 4. The first holding means 4 is fixedly connected to the carrier 2a and substantially serves to keep the position of the shaping device 2 as constant as possible with respect to the nose in that a mutual slipping is avoided. The second holding means 6 is made as an elastic band, for example as a rubber band, which engages via the fastening point 6a at the pressing part 2c so that the acting force acts substantially on the pressing surface 2d. The second holding means 6 is preferably individually adjustable so that the force acting on the pressing surface 2d can be adjusted via the second holding means 6. The second holding means 6, made as a rubber band, for example, is also placed around the head so that it also extends around the head at the rear like the first holding means 3.

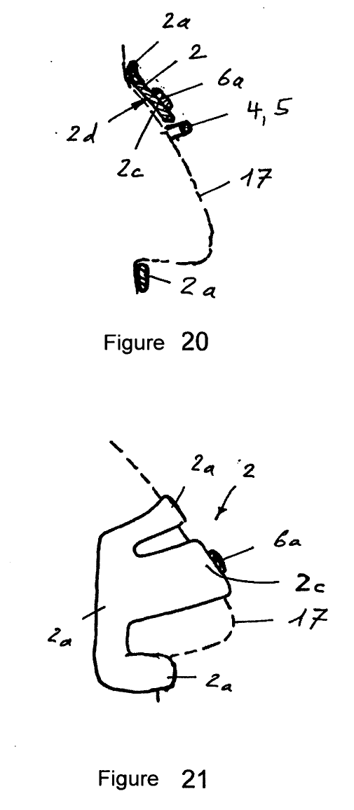

FIG. 20 shows a longitudinal section through FIG. 19 along the line A-A. The nose 17 is shown schematically. The shaping device 2 has a carrier 2a which extends to run along the root of the nose, with the carrier 2a shown at the bottom being arranged to extend between the alae of the nose and the upper lip. The pressing part 2c is part of the one-piece shaping device 2 and forms the pressing surface 2d which, in the embodiment shown, is arranged directly beneath the fastening point 6a.

FIG. 21 shows in a side view a further embodiment of a shaping device 2 with a carrier 2a, with the carrier 2a, as shown, extending along the root of the nose, with the exception of the upper section which extends, as shown, over the nose 17. The pressing part 2c is again arranged directly beneath the fastening 6a. The shaping device 2 shown in FIG. 21 is made as an injection molded part and has the advantage that it can be made in a variety of embodiments. A plurality of shaping devices 2 can, for example, be manufactured which are suitable for different nose sizes and/or nose shapes. In addition, for example, a plurality of shaping devices 2 can be manufactured whose pressing parts 2c are each arranged at different locations.

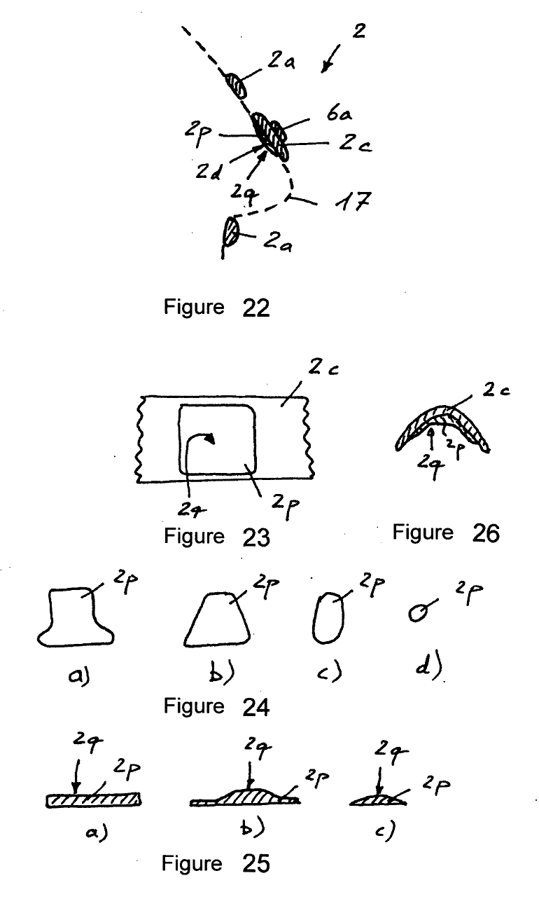

FIG. 22 shows a longitudinal section through the shaping device 2 shown in FIG. 21. The pressing part 2c has to be arranged in each case in the shaping device 2 such that it is located at that point of the nose at which a force should be effected onto the nose. It is therefore of advantage to have a plurality of shaping devices 2 whose pressing parts 2c are arranged differently so that the respectively suitable one can be selected. It is, however, naturally also possible to produce a shaping device 2 adapted individually to a nose.

FIG. 26 shows a section through an embodiment of a pressing part 2c extending perpendicular to the plane of view in accordance with FIG. 22. The extent of the curvature, the opening angle as well as the wall thickness of the pressing part 2c can be varied in a plurality of possibilities. Shaping devices 2 having a plurality of different geometrical designs could therefore be provided in order to select the best suitable shaping device 2 with respect to geometry on the fitting of the shaping device 2 to an individual nose shape. The shaping device 2 could, however, in each case also be produced with an individual geometry in accordance with the individual nose shape which is to be corrected.

As shown in FIG. 22, a cover 2p can be adhered to the shaping device 2, at the bottom at the pressing part 2c, said cover forming the support surface 2d via which the force is preferably introduced into the nose. In an advantageous embodiment, a kit is provided including at least one shaping device 2 as well as a plurality of covers 2p which, as shown in the FIGS. 24a, 24b and 24c can differ with respect to geometry and/or area and, as in the sections shown in FIGS. 25a, 25c and 25c, with respect to thickness. The cover 2p is preferably provided at the one side with an adhesive so that a suitable cover 2p can be selected and adhered to the pressing part 2c. The size, the position and the area of the introduced force is also determined, beside the geometrical extent of the pressing part 2c, by the corresponding selection of the cover 2p. In an advantageous embodiment, the cover 2p has a substantially constant thickness and is designed, for example, similar to a patch, and is made, for example, of plastic. The cover 2p could, however, also be designed like the shaping part 17k shown in FIG. 17 and, on the one hand, have a substantially areal adhesive surface and, on the other hand, have a three-dimensionally extending support surface 2d.

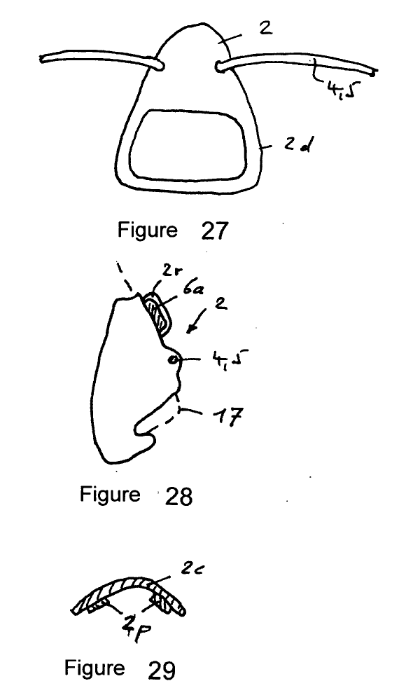

A plurality of covers 2p having differently extending support surfaces 2d and/or having different thicknesses are advantageously available for the fitting of the shaping device 2 to the nose of a patient so that the most suitable cover 2p can be adhered into the shaping device 2. Such a kit also has the advantage, as can also be seen from the section in accordance with FIG. 26, that the shaping device 2 can be adapted very individually to a nose 17, in particular also the location of the force introduction, the area of the force introduction as well as the strength of the introduced force. This has the consequence that the shaping device 2 in accordance with the invention causes less pain and also enables pleasant comfort in wear in the long term. This long-term pleasant comfort in wear is of central importance since only a regular and longer wearing of the shaping device 2 has the result that the shape of the nose or of the bone changes. The kit in accordance with the invention including at least one individually selected or individually adapted shaping device 2 as well as a plurality of geometrically different covers 2p has the substantial advantage that the cover 2p fastened in the shaping device 2 can be replaced individually and fast at any time, e.g. if an unpleasant pressure point arises or if an otherwise unpleasant wearing feeling develops. It is thereby ensured, on the one hand, that the wearing comfort is ensured in the long term and it is ensured, on the other hand, that the pressure point is not excessively strained in the long term because the pressure point is individually adjustable and also individually changeable. This individual adjustment, which can be carried out by the respective individual himself, produces the decisive advantage that the shaping device 2 in accordance with the invention is worn regularly, in the long term and with a high comfort in wear. This is a substantial advantage since only the regular and long-term wearing of the shaping device 2 ensures that the nose is shaped to the desired geometry. In addition, a continuously ideal force introduction onto the nose can be achieved in that, after a slight shaping of the nose, a different cover 2p is used, usually a thicker cover 2p. it is thereby ensured that in each case the force advantageous or ideal for the shaping of the nose is applied to the nose, which can in particular have the consequence that the shaping device 2 does not have to be worn all that long until the nose has the desired geometrical shape. FIG. 27 shows a similar shaping device 2 to that shown in FIG. 19 in which the connection means 4, 5 each extends partly within the shaping device 2.

FIG. 28 shows, similar as to in FIG. 21, a side view of a further embodiment of a shaping device 2, with the connection means 4, 5, as shown, extending at least partly within the shaping device 2. In addition, the shaping device 2 includes an eyelet 2r which is fastened to the surface of the shaping device and through which the holding means, in the embodiment shown a rubber band 6a, extends.

FIG. 29 shows, in a similar manner to FIG. 26, a section through the shaping device 2 with covers 2p arranged on its inner side. Unlike the embodiment in accordance with FIG. 26, the embodiment shown in FIG. 29 has two separate covers 2p arranged spaced apart.

The kit in accordance with the invention is commercially particularly interesting because usually an individually adapted shaping device 2 is sufficient for the total treatment, whereas a plurality of geometrically different covers 2p are required and are provided to adapt the force introduction to the nose being shaped or to the respective geometrical shape of the shaping device 2. The kit in accordance with the invention is therefore adaptable to the individual needs to a high degree and nevertheless very cost-effectively since the covers 2p can be manufactured very cost-effectively.

1. A device (1) for shaping bones, including:
a shaping device (2) having a pressing part (2c) which has a pressing surface (2d) designed for placing onto the skin;
and a holding device (3) which is connectable to the shaping device (2);
wherein the holding device (3) as well as the shaping device (2) are configured to cooperate such that the shaping device (2) can contact the skin at least at the pressing surface (2d) while exerting a pressing force, characterized in that the shaping device (2) includes a carrier (2a) having a support surface (2b); and in that the carrier (2a) is made to extend such that the support surface (2b) follows the outer contour of the root of the nose at least along a part section and can lie on the face over the support surface (2b).

2. A device in accordance with claim 1, characterized in that the carrier (2a) is configured to extend such that the support surface (2b) can lie on the face between the upper lip and the alae of the nose.

3. A device in accordance with claim 1, characterized in that the carrier (2a) is configured to extend such that the support surface (2b) surrounds the total nose and in particular extends along the root of the nose.

4. A device in accordance with claim 1, characterized in that a connection element (4) is fastened to the carrier (2a), and in that the connection element (4) can be connected to the holding device (3).

5. A device in accordance with claim 4, characterized in that the pressing part (2c) is connected to a second holding device (6).

6. A device in accordance with claim 5, characterized in that the second holding device (6) is made as an elastic band.

7. A device in accordance with claim 1, characterized in that the pressing part (2c) is journaled displaceably and optionally at least one of replaceably and fixably with respect to the carrier (2a).

8. A device in accordance with claim 1, characterized in that the carrier (2a) includes at least one holding element (2e); and in that the pressing part (2c) is connected to the holding element (2e).

9. A device in accordance with claim 8, characterized in that the holding element (2e) is made in U shape and is connected to the carrier (2a) at both ends.

10. A device in accordance with claim 8, characterized in that the holding element (2e) is made in grid shape.

11. A device in accordance with claim 1, characterized in that the pressing part (2c) includes a shaping part (2k) which forms the pressing surface (2d).

12. A device in accordance with claim 1, characterized in that the shaping device (2) is made in one piece.

13. A device in accordance with claim 1, characterized in that the pressing surface (2d) is arranged and configured in the shaping device (2) such that the pressing surface (2d) has contact at least over a part section along the bridge of the nose.

14. A device in accordance with claim 1, characterized in that the shaping device (2) is connected via an additional facial arch (5) to the holding device (3).

15. A method for manufacturing a device in accordance with claim 1 including the steps:
a) obtaining a digital model of the extent of the skin surface of the body point to be modified;
b) obtaining a digital model of the extent of the bone disposed thereunder;
c) fixing the regions of the bone to be modified;
d) determining at least one pressing surface (2d) with respect to at least one parameter from the group consisting of size, alignment, and surface extent;
e) and preparing the shaping device (2) including the pressing surface (2d).

16. A kit for the preparation of a shaping device (2) in accordance with claim 1, including at least one carrier (2a) and a plurality of differently configured pressing parts (2c) which are connectable to the carrier (2a).

17. A kit for the preparation of a one-piece shaping device (2) in accordance with claim 1 including at least one cover (2p) having a support surface (2q), with the cover (2p) being connectable to the shaping device (2) such that the support surface (2q) forms the pressing surface (2d).

18. A kit in accordance with claim 17, characterized in that the cover (2p) is made similar to a patch in that the one side of the cover (2p) forms the support surface (2q) and the other side is provided with adhesive which serves for the fastening of the cover (2p) to the shaping device (2).

19. A kit in accordance with claim 17 including a plurality of geometrically differently configured covers (2p).

20. A kit in accordance with claim 19, characterized in that the covers (2p) have different thicknesses; and optionally in that their support surfaces (2q) have at least one of different geometries and different total areas.
